(19) United States
(12) Patent Application Publication
(10) Pub. No.: US 2020/0146969 A1
(43) Pub. Date: May 14, 2020

(54) DEAD SEA SALTS COSMETIC COMPOSITIONS

(71) Applicant: Seacret Spa Ltd., Tel Aviv (IL)

(72) Inventors: Isabelle AFRIAT STALOFF, Maale Adumim (IL); Clara KOREN, Kyriat Motzkin (IL)

(73) Assignee: Seacret Spa Ltd., Tel Aviv (IL)

(21) Appl. No.: 16/612,868

(22) PCT Filed: Jul. 25, 2018
(86) PCT No.: PCT/IL18/50829

§ 371 Date: Nov. 12, 2019

Related U.S. Application Data
(60) Provisional application No. 62/540,714, filed on Aug. 3, 2017.

(51) Int. Cl. A61K 8/96; A61K 8/92; A61Q 19/10; A61Q 5/12; A61Q 5/02

(52) U.S. Cl. CPC A61K 8/965; A61K 8/922; A61Q 5/02; A61Q 5/12; A61Q 19/10

(57) ABSTRACT

Disclosed is a hydrophilic cosmetic composition comprising a surfactant and Dead Sea salts in the form of solid granules. The surfactant may include a mixture of an anionic surfactant and an amphoteric surfactant or a mixture of a non-ionic surfactant and an amphoteric surfactant. Disclosed is a hydrophilic cosmetic composition comprising a surfactant and Dead Sea salts in the form of solid granules. The surfactant may include a mixture of an anionic surfactant and an amphoteric surfactant or a mixture of a non-ionic surfactant and an amphoteric surfactant.

### FIELD OF THE INVENTION

The present invention relates to cosmetic compositions for skin exfoliation and cleansing. The compositions of the invention comprise salt granules, particularly Dead Sea salt granules, and are stable under storage and usage conditions.

### BACKGROUND OF THE INVENTION

Many cosmetic compositions comprise either both or at least one of water-insoluble granules and water soluble granules. The water insoluble granules may be synthetic granules, e.g., polyethylene spherules, or natural granules, derived from e.g., bamboo, date kernels, apricot kernels and the like. Generally, such insoluble granules are inert and are removed from the user's skin by rinsing. The water soluble granules are usually salts, such as sea salt, salts from the Himalaya's, Dead Sea salts and the like. Such salts dissolve while rinsing.

Dead Sea salts are known for their therapeutic benefits. Many known compositions comprise both salts, such as Dead Sea salts, and oily components. Some compositions further comprise surfactants, allowing the skin to be both cleansed and exfoliated. Known oil-based compositions may contain between about 30-90% salts.

Since oil-based compositions are less appealing to some consumers, due to their greasy texture, and since oil-based compositions generally do not exhibit desired foaming properties, hydrophilic cleansing compositions containing Dead Sea salts have been developed. Such compositions generally include Dead Sea salts in non-granular form, wherein the granular components in those compositions are prepared from other salts and/or insoluble materials. Nonetheless, many of these products are either not stable, do not have the required foaming properties, and have further disadvantages.

Moreover, it is known that while the addition of surfactants is advantageous, many surfactants, including anionic surfactants, such as, sodium cocoyl glycinate and sodium lauroyl glutamate, are incompatible with Dead Sea salts. There is a need for a formulation with Dead Sea Salts and surfactants for cosmetic purposes.

### SUMMARY OF THE INVENTION

In an embodiment of the invention, there is provided a hydrophilic cosmetic composition comprising a surfactant and Dead Sea salts in the form of solid granules. In some embodiments of the invention, the surfactant includes a mixture of an anionic surfactant derived from taurate, isethionate, sulfonate and/or sulfosuccinate or any combination thereof and an amphoteric surfactant derived from at least one betain or lauroamphoacetate.

In some embodiments of the invention, the surfactant includes a mixture of a non-ionic surfactant derived from alcohol, alkanolamide, ester or amine oxide or any combination thereof; and an amphoteric surfactant derived from at least one betain or lauroamphoacetate.

In some embodiments of the invention, the weight ratio between the amphoteric surfactant and the non-ionic surfactant is in the range of between about 50/50 and 80/20.

In some embodiments of the invention, the weight ratio between the amphoteric surfactant and the ionic surfactant is in the range of between about 2:1 and 3:1.

In some embodiments of the invention the solid Dead Sea salt granules have an average diameter of between about 0.1 and 0.5 mm.

In some embodiments of the invention, the composition comprises at least 20%, 30%, 40%, 50%, 60%, 70% or 80% w/w Dead Sea salts in the form of granules.

In some embodiments of the invention, the composition further includes glycerin, propane-diol, propylene glycol, butylene glycol and/or a polyglycol.

In some embodiments of the invention the composition further comprises an essential oil. In some embodiments of the invention, the essential oil is an oil commonly used in cosmetic, such as without limitation, peppermint oil, orange essential oil, lavender oil, jasmin oil and the like.

In some embodiments, the composition further contains a gelling agent such as PVP (polyvinylpyrrolydone) or cellulose derived gelling agents such as hydroxyethylcellulose or xanthan gum or polyacrylate such as polyacrylate crosspolymer-6 or a mixture thereof.

According to some embodiments of the invention, the composition is in a form of a liquid gel, shampoo, cream, a shower gel, a hair lotion, an exfoliating gel or a soap.

### DETAILED DESCRIPTION OF THE INVENTION

It is noted that throughout, all ranges and numeric figures are considered to be approximate even when the term “about” is not used, such that the document is considered to cover ±10% of the disclosed range or figure, unless specifically mentioned otherwise. It is further noted that the term “about” is also considered to cover ±10% of the disclosed range or figure, unless specifically mentioned otherwise.

Some embodiments of the invention are directed to a hydrophilic cleansing and/or exfoliating foaming and stable composition that comprises Dead Sea salt granules and a surfactant. In some embodiments of the invention there is provided a hydrophilic cleansing and/or exfoliating foaming and stable composition that comprises Dead Sea salt granules and a mixture of two or more surfactants. According to some embodiments of the invention, the surfactant mixture is composed of one or more amphoteric surfactants together with one or more non-ionic and/or one or more ionic surfactants.

In some embodiments of the invention, the amphoteric surfactant is one or more members of the betain family. In some embodiments, the amphoteric surfactant is lauroamphoacetate.

In some embodiments, the weight ratio between the amphoteric surfactant and the non-ionic surfactant is in the range of between about 50/50 and 80/20.

In some embodiments, the weight ratio between the amphoteric surfactant and the ionic surfactant is in the range of between about 2:1 and 3:1.

In some embodiments of the invention, the ionic surfactant is one or more of taurate, isethionate, sulfonate and sulfosuccinate

In some embodiments of the invention, the non-ionic surfactant is one or more of an alcohol, alkanolamide, ester and amine oxide. In some embodiments, the alcohol is cetyl or stearyl alcohol. In some embodiments, the amine oxide is cocamodopropylamine oxide and the like.

According to some embodiments, the composition comprises at least about 20% w/w Dead Sea salts in the form of granules. According to some embodiments, the composition comprises at least about 30% w/w Dead Sea salts in the form of granules. According to some embodiments, the composition comprises at least about 40% w/w Dead Sea salts in the form of granules. According to some embodiments, the composition comprises at least about 50% w/w Dead Sea salts in the form of granules. According to some embodiments, the composition comprises at least about 60% w/w Dead Sea salts in the form of granules. According to some embodiments, the composition comprises at least about 70% w/w Dead Sea salts in the form of granules. According to some embodiments, the composition comprises at least about 80% w/w Dead Sea salts in the form of granules.

According to some embodiments, the composition comprises between about 45-90% w/w Dead Sea salts in the form of granules. According to some embodiments, the composition comprises between about 55-85% w/w Dead Sea salts in the form of granules.

According to some embodiments, the size of the Dead Sea salt granules is such that the maximum linear dimension of the salt granules is between about 0.1-0.5 mm According to some embodiments, the size of the Dead Sea salt granules is such that the maximum linear dimension of the salt granules is between about 0.1-0.2 mm According to some embodiments, the size of the Dead Sea salt granules is such that the maximum linear dimension of the salt granules is between about 0.2-0.3 mm According to some embodiments, the size of the Dead Sea salt granules is such that the maximum linear dimension of the salt granules is between about 0.3-0.4 mm According to some embodiments, the size of the Dead Sea salt granules is such that the maximum linear dimension of the salt granules is between about 0.4-0.5 mm.

In some embodiments, the Dead Sea salt composition comprises at least one of the minerals selected from chloride, magnesium, sulfate, potassium, sodium, calcium, bicarbonate, lithium, boron, nitrogen, e.g., in the form of ammonia, fluoride, aluminium, silicon, manganese, iron, copper, zinc, bromide, rubidium, strontium and barium.

According to some embodiments, the composition of the invention may be a cleansing composition, an exfoliating composition, or both. The compositions may be formulated as a facial composition, body composition, shampoo, mask, cream, including scalp cream, and the like.

According to some embodiments, the compositions contain at least one oil selected from the group of vegetable oils, esters, silicone oils and any combination thereof. The compositions can contain active ingredients such as vitamins, peptides, acids, glycans, glycosaminoglycans, plant extracts, dead sea mud, polysaccharides, alcohol, and other known active ingredients for skin and/or hair improvement.

In some embodiments, the formulation further comprises one or more of glycerin, propane-diol, propylene glycol, butylene glycol and/or other types of polyglycol. In some embodiments of the invention the amount of the one or more of glycerin, propane-diol, propylene glycol, butylene glycol and/or other types of polyglycol is between 0.1 to 50% w/w. In some embodiments of the invention the amount of the one or more of glycerin, propane-diol, propylene glycol, butylene glycol and/or other types of polyglycol is between 0.1 to 10% w/w. In some embodiments of the invention the amount of the one or more of glycerin, propane-diol, propylene glycol, butylene glycol and/or other types of polyglycol is between 1 to 50% w/w. In some embodiments of the invention the amount of the one or more of glycerin, propane-diol, propylene glycol, butylene glycol and/or other types of polyglycol is between 10 to 50% w/w. In some embodiments of the invention the amount of the one or more of glycerin, propane-diol, propylene glycol, butylene glycol and/or other types of polyglycol is between 2 to 50% w/w. In some embodiments of the invention the amount of the one or more of glycerin, propane-diol, propylene glycol, butylene glycol and/or other types of polyglycol is between 30 to 50% w/w.

In some embodiments of the invention the composition further comprises an essential oil. In some embodiments of the invention, the essential oil is an oil commonly used in cosmetic, such as without limitation, peppermint oil, orange essential oil, lavender oil, jasmin oil and the like. In some embodiments of the invention, the amount of the essential oil is between 0.01 to 5% w/w. In some embodiments of the invention, the amount of the essential oil is between 0.01 to 3% w/w. In some embodiments of the invention, the amount of the essential oil is between 0.01 to 2% w/w. In some embodiments of the invention, the amount of the essential oil is between 0.01 to 1% w/w. In some embodiments of the invention, the amount of the essential oil is between 0.01 to 0.1% w/w.

According to some embodiments, the compositions may include any additional number of ingredients, including conditioning agents, thickeners, polymers or any combination thereof. According to some embodiment, the formulation further comprises at least one fragrance, essential oil, plant extract, hydrogenated oil, vitamin, peptide, polyphenol, polysaccharide, gelling agent, conditioning agent such as cationic polymers, or any combination thereof.

In a particular embodiment, the composition can include at least one vegetable oil, ester, non polar oil, or any combination thereof.

The following examples are given by way of illustration for the preparation of compositions, their testing and methods of administering the same, according to various embodiments of the invention. However, these particular examples are for purposes of illustration and are not intended to limit the scope of the invention.

### EXAMPLES

### Example 1

TABLE Ia
Facial exfoliator cleanser composition

| Component | Amount (% w/w) |
|---|---|
| water | 8 |
| glycerine | 19 |
| Lauroyl/Myristoyl Methyl Glucamide | 6 |
| 25%; (Coco-Betaine 22.5%; and Water | |
| 52.5% (GlucoTain Liquiflex) | |
| sodium lauroamphoacetate | 1 |
| Dead Sea salts (granules) | 65 |
| Fragrance or an essential oil | 1 |

TABLE Ib
Facial exfoliator cleanser composition

| Component | Amount (% w/w) |
|---|---|
| water | 5-15 |
| glycerine | 10-25 |
| Lauroyl/Myristoyl Methyl Glucamide 25%; | 3-9 |
| (Coco-Betaine 22.5%; and Water 52.5% | |
| (GlucoTain Liquiflex) | |
| sodium lauroamphoacetate | 0.5-4 |
| Dead Sea salts (granules) | 40-70 |
| Fragrance or an essential oil | 0.5-3 |

TABLE Ic
Facial exfoliator cleanser composition

| Component | Amount (% w/w) |
|---|---|
| water | 2-20 |
| glycerine | 7-30 |
| Lauroyl/Myristoyl Methyl Glucamide 25%; | 1-13 |
| (Coco-Betaine 22.5%; and Water 52.5% | |
| (GlucoTain Liquiflex) | |
| sodium lauroamphoacetate | 0.3-6 |
| Dead Sea salts (granules) | 40-70 |
| Fragrance or an essential oil | 0.3-4 |

### Preparation Procedure

Water and glycerin were mixed together to form a water-glycerin solution. A pre-mix of the fragrance and 10% of the surfactant mixture was prepared separately. The remaining 90% of the surfactant mixture was added to the water-glycerin solution, thereby obtaining a water-glycerin-surfactant solution. The pre-mix fragrance-surfactant solution and the Dead Sea salts were then added slowly to the water-glycerin-surfactant solution until a homogeneous solution was obtained.

### Example 2

TABLE IIa
Exfoliating scalp mask treatment composition

| Component | Amount (% w/w) |
|---|---|
| Water | 2 |
| Coco-Betaine 24%; | 9 |
| Sodium Methyl Cocoyl Taurate 6.5%; | |
| Sodium Cocoyl Isethionate 5.5%; | |
| and Water (Genagen 3SB) | |
| Polyquaternium-10 | 1 |
| Coco-Betain | 4 |
| Xanthan gum | 1 |
| Peppermint essential oil | 0.2 |
| Dead Sea salts (granules) | 82.8 |

TABLE IIb
Exfoliating scalp mask treatment composition

| Component | Amount (% w/w) |
|---|---|
| Water | 1-3 |
| Coco-Betaine 24%; | 6-12 |
| Sodium Methyl Cocoyl Taurate 6.5%; | |
| Sodium Cocoyl Isethionate 5.5%; | |
| and Water (Genagen 3SB) | |
| Polyquaternium-10 | 0.5-3 |
| Coco-Betain | 2-5 |
| Xanthan gum | 0.2-3 |
| Peppermint essential oil | 0.05-0.4 |
| Dead Sea salts (granules) | 70-85 |

TABLE IIc
Exfoliating scalp mask treatment composition

| Component | Amount (% w/w) |
|---|---|
| Water | 0.5-4 |
| Coco-Betaine 24%; | 5-15 |
| Sodium Methyl Cocoyl Taurate 6.5%; | |
| Sodium Cocoyl Isethionate 5.5%; | |
| and Water (Genagen 3SB) | |
| Polyquatemium-10 | 0.3-5 |
| Coco-Betain | 1-7 |
| Xanthan gum | 0.1-5 |
| Peppermint essential oil | 0.03-0.7 |
| Dead Sea salts (granules) | 70-90 |

Three subjects with scalp itchiness tried the product and felt significant relief after one use, which lasted for 1-2 days.

### Example 3

The purpose of the experiments was to demonstrate that formulating a cleansing hydrophilic composition with Dead Sea minerals requires different surfactants than a cleansing composition containing sodium chloride. Trials 1, 2 and 3 were performed with NaCl, whereas trials 4, 5 and 6 were conducted with Dead Sea salt granules. Known surfactants were added to each of the compositions according to the table below:

| | TRIAL | TRIAL | TRIAL | TRIAL | TRIAL | TRIAL |
|---|---|---|---|---|---|---|
| Ex./agent | 1 | 2 | 3 | 4 | 5 | 6 |
| HOSTAPON | 33 | — | 16 | 33 | — | 16 |
| SG | | | | | | |
| (CLARIANT) | | | | | | |
| Sodium | | | | | | |
| cocoyl | | | | | | |
| glycinate (%) | | | | | | |
| PLANTAPON | — | 33 | 16 | — | 33 | 16 |
| SCG/SG | | | | | | |
| (CLARIANT) | | | | | | |
| Sodium | | | | | | |
| lauroyl | | | | | | |
| glutamate (%) | | | | | | |
| NaCl granules | 67 | 67 | 68 | — | — | — |
| (%) | | | | | | |
| Dead sea salt | — | — | — | 67 | 67 | 68 |
| granules (%) | | | | | | |

Compositions containing sodium chloride granules in high amounts such as 30 to 40% are known in the art. Such compositions contain anionic surfactants pertaining to the family of sodium cocoyl glycinate and sodium lauroyl glutamate. These surfactants or combination of surfactants were not compatible with dead sea salts.

The incompatibility was determined according to the homogenicity of the prepared compositions. The results showed that the compositions in trials 1, 2 and 3 were homogeneous blends whereas compositions 4, 5 and 6 were not homogeneous; rather, two separate phases were formed. Thus, it is clear from the presented results that the surfactants required for compositions comprising Dead Sea salts are different than those used in compositions comprising NaCl.

It will be understood by those skilled in the art that various changes in form and details may be made therein without departing from the spirit and scope of the invention as set forth in the appended claims. Those skilled in the art will recognize, or be able to ascertain using no more than routine experimentation, many equivalents to the specific embodiments of the invention described herein. Such equivalents are intended to be encompassed in the scope of the claims.

1. A hydrophilic cosmetic composition comprising a surfactant and Dead Sea salts in the form of solid granules, wherein the surfactant includes a mixture of an anionic surfactant and an amphoteric surfactant.

2. The hydrophilic cosmetic composition according to claim 1, wherein the anionic surfactant is derived from taurate, isethionate, sulfonate, sulfosuccinate or any combination thereof; the amphoteric surfactant is derived from at least one betain or lauroamphoacetate, or combination thereof.

3. The hydrophilic cosmetic composition according to claim 1, wherein the surfactant further includes a mixture of a non-ionic surfactant derived from alcohol, alkanolamide, ester, amine oxide or any combination thereof.

4. The hydrophilic cosmetic composition according to claim 3, wherein the weight ratio between the amphoteric surfactant and non-ionic surfactant is in the range of between 50/50 and 80/20.

5. The hydrophilic cosmetic composition according to claim 1, wherein the weight ratio between the amphoteric surfactant and anionic surfactant is in the range of between 2:1 and 3:1

6. The hydrophilic cosmetic composition according to claim 1, wherein the solid Dead Sea salt granules have an average diameter of between 0.1 mm and 0.5 mm.

7. The hydrophilic cosmetic composition according to claim 1, wherein said composition comprises at least 20% w/w of Dead Sea salts in the form of granules.

8. The hydrophilic cosmetic composition according to claim 1, wherein the composition further includes glycerin, propane-diol, propylene glycol, butylene glycol, a polyglycol, or any combination thereof.

9. The hydrophilic cosmetic composition according to claim 1, wherein the composition further comprises an essential oil.

10. The composition of claim 1, wherein the composition is in a form of a liquid gel, shampoo, cream, a shower gel, a hair lotion, or an exfoliating gel or a soap.
